(19) United States
(12) Patent Application Publication
(10) Pub. No.: US 2023/0212617 A1
(43) Pub. Date: Jul. 6, 2023

(54) METHODS AND PREPARATION FOR HEMP BIODEGRADABLE ABSORBENT

(71) Applicant: Organa Dry LLC, Bedford, KY (US)

(72) Inventors: Evan Ogburn, Bedford, IN (US); John Ogburn, Milton, KY (US); Chris Yates, Milton, KY (US); Michael Yates, Wilder, KY (US)

(73) Assignee: Organa Dry LLC, Bedford, KY (US)

(21) Appl. No.: 18/090,881

(22) Filed: Dec. 29, 2022

Related U.S. Application Data
(60) Provisional application No. 63/295,181, filed on Dec. 30, 2021.

(51) Int. Cl. C12P 7/10

(52) U.S. Cl. CPC C12P 7/10; C12P 2201/00

(57) ABSTRACT

A hemp biodegradable absorbent according to various aspects of the present technology comprises receiving hemp biomass and processing the biomass into a desired particle range through a separation process. The separation process may comprise an initial filtration to separate out particles having a size of greater than 500 μm, pulverizing or sifting the remaining particles into a plurality of grades in a range of 0.5 μm to 50 μm, and selecting at least one grade of particles to form the absorbent. The selected particles are dried to a moisture content of less than about 10% to create a hemp biodegradable absorbent base that can be used to form various types of absorbent products that may provide an absorbent capacity of between about at least about 300% and up to about 600% by weight. The hemp biodegradable absorbent may also be effective at absorbing both oil-based and water-based liquids.

### CROSS-REFERENCES TO RELATED APPLICATIONS

This application claims the benefit of U.S. Provisional Patent Application No. 63/295,181, filed on Dec. 30, 2021, and incorporates the disclosure of this application by reference.

### BACKGROUND OF THE TECHNOLOGY

Within many industries, oil, water, glue, solvents, and other chemical spills occur frequently. Absorbent powders are typically applied to contain and clean up those spills within locations such as manufacturing plants. Typical absorbents are commonly heavy compounds that made from, for example, crushed rock. This is an issue because shipping costs are high due to product weight, and more importantly, the material is difficult and heavy to clean up after it is saturated with the liquid. Because of this a scoop shovel and bucket are needed for disposal, increasing cost, time, and effort associated with cleanup of the spill. Another downside to typical industrial absorbents is that they are not efficient at soaking up either oil or water. Some do well with one but not the other. For example, certain types of absorbents may take an hour or more to soak up water. This is inconvenient and cost prohibitive. Finally, this product and production process provides a solution to a problem in the hemp industry. Hemp biomass extractors in the US, and throughout the world, are having to dispose of their extracted biomass at landfills, creating additional waste and expense for the extractors.

### SUMMARY OF THE TECHNOLOGY

A hemp biodegradable absorbent according to various aspects of the present technology comprises receiving hemp biomass and processing the biomass into a desired particle range through a separation process. The separation process may comprise an initial filtration to separate out particles having a size of greater than 500 μm, pulverizing or sifting the remaining particles into a plurality of grades in a range of 0.5 μm to 50 μm, and selecting at least one grade of particles to form the absorbent. The selected particles are dried to a moisture content of less than about 10% to create a hemp biodegradable absorbent base that can be used to form various types of absorbent products that may provide an absorbent capacity of between about at least about 300% and up to about 600% by weight. The hemp biodegradable absorbent may also be effective at absorbing both oil-based and water-based liquids.

### BRIEF DESCRIPTION OF THE DRAWINGS

A more complete understanding of the present technology may be derived by referring to the detailed description when considered in connection with the following illustrative figures. In the following figures, like reference numbers refer to similar elements and steps throughout the figures.

FIG. 1 representatively illustrates a flowchart of a process for forming a hemp biodegradable absorbent in accordance with an exemplary embodiment of the present technology;

FIG. 2 representatively illustrates a blending process for a hemp biodegradable absorbent product in accordance with an exemplary embodiment of the present technology; and

FIG. 3 representatively illustrates a flowchart of a process for forming a hemp biodegradable absorbent in accordance with an exemplary embodiment of the present technology.

### DETAILED DESCRIPTION OF EXEMPLARY EMBODIMENTS

The present technology may be described in terms of functional block components and various processing steps. Such functional blocks may be realized by any number of components configured to perform the specified functions and achieve the various results. For example, the present technology may employ various types of processing methods for manufacturing the biodegradable absorbent. In addition, the present technology may be practiced in conjunction with any number of extracted byproducts, and the system described is merely one exemplary application for the technology. Methods and preparation for a biodegradable absorbent according to various aspects of the present technology may operate in conjunction with any processed hemp materials resulting from various types of extraction processes. The present technology describes a process that converts any form of used, processed hemp, regardless of the extraction method, into a uniform, dried material, of known particle size and composition, into a biodegradable absorbent.

Currently, there are many processes available to extract fluid from hemp and to process that hemp material prior to and post-extraction. Specifically, fluid extraction processes used to process hemp leave the physical form of the hemp substantially unaltered as compared to extraction processes that use pressing mechanisms which ultimately crushes the subject material. Consequently, depending on the type of process used, the leftover hemp may retain or possess any number of desirable qualities such as the ability to absorb fluids.

For example, a number of suitable extraction processes known as liquid-liquid extraction or solvent extraction are effective for extracting hemp for the purposes of producing cannabinoid containing extracts such as CBD oil. These extraction processes include options such as: supercritical fluid extraction (using supercritical carbon dioxide (CO2)), conventional solvent extraction, subcritical CO2 extraction, and carrier oil extraction. Conventional extraction solvents associated with conventional solvent extraction may include grain alcohol, ethanol high grade alcohol, butane, isopropyl alcohol or, hexane while the carrier oil extraction process may utilize olive oil. Subcritical or supercritical CO2 extraction process use CO2 that is non-toxic and non-flammable as compared to the chemicals employed in solvent extraction. Subcritical or supercritical CO2 extraction also removes the beneficial plant waxes and the chlorophyll from the biomass.

Depending on the end use, the specifically chosen process for extracting the oil may vary. For example, conventional solvent extraction may be less expensive and easier to perform over supercritical CO2 extraction.

As a result of the CBD oil extraction process from hemp, used processed hemp biomass is produced. This material is deprived of the biomolecules and oleoresin that is contained naturally within the hemp plant. When the physical form of the processed hemp is left largely unaltered, hemp biomass can absorb liquids up to 490% of its weight. Depending on the particle size of the hemp biomass material, and the extraction process used to process the hemp biomass material, the absorption capacity may vary in unexpected ways.

The disclosed technology is directed towards generating a more uniformly sized hemp biomass material that is suitably configured to absorbs liquids more quickly and in higher quantity than materials of varying particle size. For example, in one embodiment, the processed hemp biomass material may comprise particles having a specified average size generally between about 0.5 um and about 10 cm with a desired moisture content. The particle size and/or shape may be selected according to a desired absorption rate or capacity such that the surface area may be matched according to a desired absorption capacity. It has been found that increases in surface area of individual particles of the hemp biomass material relative to particle weight provides greater absorption capacity as compared to absorbent materials having larger particle sizes.

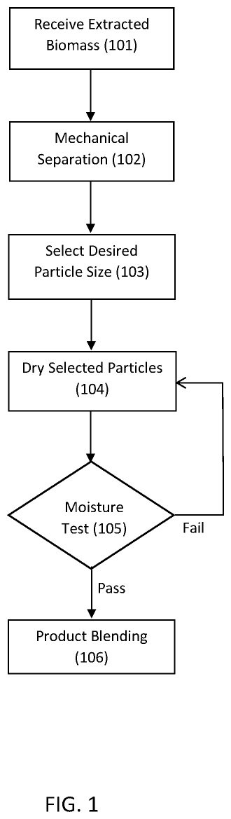

Referring now to FIG. 1, a process for producing a hemp biodegradable absorbent may comprise receiving hemp byproduct (hemp biomass) from a hemp extraction process. The hemp biomass may be devoid of oils, resins, biomolecules, waxes, lipids, chlorophyll and other undesirable components (101). For example, in one embodiment, the extracted hemp biomass may comprise a hemp biomass without biomolecules and oleoresins and have a moisture content of between about 5% and about 25%.

The hemp biomass may be processed to produce a desired particle range. For example, the hemp biomass may be separated into a plurality of grades by a mechanical separation process (102). Any suitable process or system may be used to process the hemp biomass to obtain the desired grade(s) of particles. In one embodiment, the mechanical separation process may include a multi-stage process incorporating both screening and mechanical processing.

For example, in one embodiment, the process may comprise an initial filtration to filter particles having a desired range of particle sizes and selecting the desired grades of particles (103). The initial filtration may comprise any suitable method for filtering particles to the desired particle range. For example, the initial filtration may separate particles having a size of greater than 0.5 μm using any suitable filter mechanism. The filtered hemp biomass material may be passed through a fine-screen hammer mill set to 0.5 μm. The selected grades of particles may comprise any suitable size. For example, in some embodiments, the hemp biomass may be screened and/or pulverized to provide a particle size of between less than or equal to 0.5 μm and up to about 10 cm.

In an alternative embodiment, the mechanical separation process may comprise an additional step of pulverization. The separation process may comprise an initial filtration separating particles having a desired range of particle sizes; pulverizing the particles having a desired range of particle sizes and selecting the desired grades of particle sizes. For example, the separation process may include an initial filtration to separate particles having a size of greater than 500 μm; pulverizing the remaining particles; sifting the pulverized particles into a desired range of grades; and selecting the desired grades of particles (103).

Selection of grades may include selection of any suitable number of grades for the intended formulation and application of the hemp biodegradable absorbent. For example, in one embodiment, the selection of grades comprises selecting grades above the threshold particle size of 10 cm. In some embodiments, the selection of grades comprises selecting grades in the range of 1 cm. In another embodiment, the selection of grades comprises selecting grades below a threshold particle size of 500 μm. In one embodiment, the selection of grades includes selecting grades in a range of between about 0.5 μm and about 50 μm.

The selected particles may be blended by any suitable method (104). The blended grades are then dried and/or stored to achieve a desired percent moisture content (104/105). The hemp biomass may be dried to any suitable moisture content depending on its intended use. For example, in one embodiment, the hemp biomass may be dried to a moisture content of less than about 10% by weight. In various embodiments, the hemp biomass is tested for moisture content at various stages of the drying process (106).

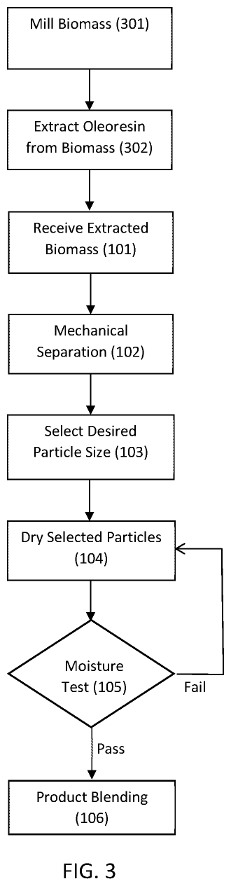

Referring to FIG. 3, in some embodiments, the process for producing a hemp biodegradable absorbent further comprises milling the hemp biomass (301) and extracting biomolecules and oleoresins from the hemp biomass to produce the hemp biomass without biomolecules and oleoresins (302). The extraction may be completed using any suitable method for removing of biomolecules and oleoresins from the hemp biomass as outlined in paragraph 12.

The hemp biomass may be formulated to produce a hemp biodegradable absorbent or other product. In one embodiment, the hemp biodegradable absorbent may be formulated to produce any suitable properties to absorb liquids such as particle size, moisture content and absorbent capacity. For example, the hemp biodegradable absorbent may consist of a particle size in the range of about 0.5 μm to about 500 μm; a moisture content of less than about 10%; and having an absorbent capacity of between about 300% and up to about 600% by weight. In one embodiment, the hemp biodegradable absorbent may consist of a particle size in the range of between 0.5 μm and about 50 μm; a moisture content of less than about 10%; and an absorbent capacity of about 480% by weight.

In testing, the hemp biodegradable absorbent shows an improved absorption capacity across a greater variety of fluids in comparison to preexisting absorption products. In One embodiment, the observed absorption capacity of a hemp biodegradable absorbent having of a particle size in the range of about 0.5 μm and a moisture content of less than about 10% by weight revealed better than expected improvements compared to a standard clay-based absorbents, synthetic absorbents, and other natural plant-based materials. For example, and referring now to Table 1, a comparison of the absorption rate of the hemp biodegradable absorbent to a prior art clay-based absorbent shows the hemp biodegradable absorbent has an improved absorption rate by volume compared to the standard clay-based absorbent for a variety of fluids. In one test of absorption of one gallon of motor oil, the hemp biodegradable absorbent absorbed 5.9 times more volume by weight than the standard clay-based absorbent. These improvements extend across various types of fluids including both oil-based fluids and water-based fluids. Not only was it shown that less hemp biodegradable absorbent is required to absorb a given fluid, but it also can be used in applications where a clay-based material can't.

TABLE 1
Absorption rate improvement comparison between hemp biodegradable
absorbent and standard clay-based absorbent.

| | Hemp | | Absorption Rate |
|---|---|---|---|
| Test Fluid | Biodegradable | Clay-based | Improvement |
| (1 gallon) | Absorbent (lbs) | Absorbent (lbs) | (volume by weight) |
| Motor Oil | 1.80 | 10.60 | 5.9 |
| Anti-Freeze | 1.90 | 13.00 | 6.8 |
| Hydraulic Fluid | 1.92 | 11.20 | 5.8 |
| Diesel Fluid | 1.98 | 10.40 | 5.3 |
| Cooking Oil | 2.20 | 11.30 | 5.1 |
| Latex Paint | 2.60 | 24.70 | 9.5 |
| Ink | 3.50 | 30.00 | 8.6 |
| Water | 5.00 | Not Applicable | |

The hemp biodegradable absorbent may be used in various applications to absorb many different types of liquids such as oil, water, chemical, and other liquid spills, either alone or simultaneously. Depending on a desired application, the hemp biodegradable absorbent may be formulated into additional forms for use in the cleanup of industrial spills comprising oils or other manufacturing related chemicals. For example, the hemp biodegradable absorbent may be formulated for use as an amendment for athletic fields to absorb water and prevent change to field conditions. In various embodiments, the hemp biodegradable absorbent may be suitably formulated for other applications such as an animal litter, absorbent pads and pallets, and/or as a biodegradable additive to fertilizers.

In some embodiments, additional ingredients may be added to the hemp biodegradable absorbent according to its end use. Any additional ingredients may be used, for example, dust-reducing agents, surfactants, clumping agents, color changing agents, coloring agents, fragrance, fertilizer, anti-microbial agents, anti-odor agents, silica, starch, clay, neutralizing compounds, brick dust, and/or sand.

In an exemplary embodiment, the hemp biodegradable absorbent may be formulated as a liquid absorbent for use in industrial spills comprising oils or other manufacturing related chemicals. For example, in one embodiment, the hemp biodegradable absorbent may be formulated with a particle size in the range of about 0.5 μm to about 500 μm having a moisture content of less than about 10% by weight and provide an absorbent capacity of up to about 480% by weight. In an alternative embodiment, the hemp biodegradable absorbent may be formulated with a consistent particle size in the range of about 0.5 μm having a moisture content of less than about 10% by weight and an absorbent capacity of about 480% by weight.

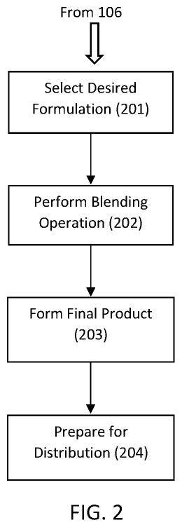

In another representative embodiment, the hemp biodegradable absorbent may be formulated as an amendment for athletic fields. For example, in one embodiment the hemp biodegradable absorbent may comprise of a particle size in the range of up to 500 μm having a moisture content of less than about 10% by weight and provide an absorbent capacity of up to about 480% by weight. With reference now to FIG. 2, once the desired formulation is selected (201), the hemp biodegradable absorbent may be blended with any suitable number of ingredients (202). For example, when blending a composition to form the amendment for athletic fields, the hemp biodegradable absorbent may be combined with additional ingredients such as dust-reducing agents, brick dust, coloring agents, and/or sand (203). The final blended product may then be prepared for distribution (204) such as by being bagged for transport and sale.

In yet another formulation, the hemp biodegradable absorbent may be formulated to absorb released bodily fluids from animals. Any suitable number of ingredients may be included in the formulation of the hemp biodegradable absorbent such as clumping agents, surfactant, dust-reducing agents, fragrance, and anti-microbial agents. The blended formulation may further undergo a processing step to be pelletized or otherwise processed into any suitable shape or consistency for absorbing bodily fluids. For example, if the hemp biodegradable absorbent is formulated into a cat litter product, the blended formulation may be formed into pellets on the order of about ½ cm to about 2 cm in length. Another formulation may result in a more sand-like consistency with particle sizes on the order of about ½ mm to about 2 mm in size.

In some embodiments, the hemp biodegradable absorbent may be formulated into a composition used as a fertilizer. Any suitable number of ingredients used a fertilizers or constituents of fertilizers may be included into the formulation of the hemp biodegradable absorbent such as nitrogen, phosphorus, potassium, manure, or any other like components.

In some embodiments, the hemp biodegradable absorbent may be formed into absorbent pads or pallets. For example, the hemp biodegradable absorbent may be used as a filler in an absorbent pad or pallet. Any suitable number of ingredients may be included in the formulation of the hemp biodegradable absorbent. For example, the hemp biodegradable absorbent may be combined with decorticated hemp fiber, hemp stalk, and/or hemp stem having a particle size of up to about 10 cm. The blended composition may also be combined with any suitable plant-based or synthetic fiber and formed into a suitable shape or size of pad or pallet.

The particular implementations shown and described are illustrative of the technology and its best mode and are not intended to otherwise limit the scope of the present technology in any way. Indeed, for the sake of brevity, conventional manufacturing, connection, preparation, and other functional aspects of the system may not be described in detail. Furthermore, the connecting lines shown in the various figures are intended to represent exemplary functional relationships and/or steps between the various elements. Many alternative or additional functional relationships or physical connections may be present in a practical system.

In the foregoing specification, the technology has been described with reference to specific exemplary embodiments. Various modifications and changes may be made, however, without departing from the scope of the present technology as set forth in the claims. The specification and figures are illustrative, rather than restrictive, and modifications are intended to be included within the scope of the present technology. Accordingly, the scope of the technology should be determined by the claims and their legal equivalents rather than by merely the examples described.

For example, the steps recited in any method or process claims may be executed in any order and are not limited to the specific order presented in the claims. Additionally, the components and/or elements recited in any apparatus claims may be assembled or otherwise operationally configured in a variety of permutations and are accordingly not limited to the specific configuration recited in the claims.

Benefits, other advantages and solutions to problems have been described above with regard to particular embodiments; however, any benefit, advantage, solution to problem or any element that may cause any particular benefit, advantage or solution to occur or to become more pronounced are not to be construed as critical, required or essential features or components of any or all the claims.

As used herein, the terms “comprise,” “comprises,” “comprising,” “having,” “including,” “includes,” or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present technology, in addition to those not specifically recited, may be varied or otherwise particularly adapted to specific environments, manufacturing specifications, design parameters or other operating requirements without departing from the general principles of the same. Any terms of degree such as “substantially,” “about,” and “approximate” as used herein mean a reasonable amount of deviation of the modified term such that the end result is not significantly changed. For example, these terms can be construed as including a deviation of at least ±5% of the modified term if this deviation would not negate the meaning of the word it modifies.

1. A process for producing a hemp biodegradable absorbent, comprising:
receiving extracted hemp biomass without biomolecules and oleoresins and having a moisture content of between about 5% and about 25%;
processing the received hemp biomass into a desired particle range, wherein processing the hemp biomass comprises:
an initial filtration to separate out particles having a size of greater than 500 μm; and
a secondary filtration to select out hemp biomass particles having a size of about 0.5 μm;
drying the selected hemp biomass particles to a moisture content of less than about 10%;
formulating the hemp biodegradable absorbent from the selected hemp biomass particles, wherein the hemp biodegradable absorbent comprises:
a particle size in the range of about 0.5 μm;
a moisture content of less than about 10%; and
an absorbent capacity of between at least about 300% and up to about 600% by weight.

2. A process for producing a hemp biodegradable absorbent according to claim 1, wherein processing the received hemp biomass into a desired particle range further comprises:
pulverizing the received hemp biomass into a plurality of grades having a particle size in a range of between about 0.5 μm and about 50 μm;
selecting at least two grades of particles from the plurality of grades, wherein at least one selected grade is about 0.5 μm in size or less; and
blending the selected grades of particles together.

3. A process for producing a hemp biodegradable absorbent according to claim 1, wherein processing the received hemp biomass into a desired particle range further comprises:
sifting the particles from the initial filtration into a plurality of grades having a particle size in a range of between about 0.5 μm and about 50 μm;
selecting at least two grades of particles from the plurality of grades, wherein at least one selected grade is about 0.5 μm in size or less; and
blending the selected grades of particles together.

4. A process for producing a hemp biodegradable absorbent according to claim 1, further comprising:
milling the hemp biomass; and
extracting biomolecules and oleoresins from the hemp biomass.

5. A process for producing a hemp biodegradable absorbent according to claim 4, wherein extracting biomolecules and oleoresins from the hemp plant to produce the hemp biomass without biomolecules and oleoresins comprises the steps of:
isolating the biomolecules and oleoresins using carbon dioxide at super-critical pressure and at a temperature range from 0° to 40° C.; and
separating the extract of biomolecules and oleoresins from the hemp biomass without biomolecules and oleoresins at sub-critical temperature and sub-critical pressure.

6. A process for producing a hemp biodegradable absorbent according to claim 1, wherein the hemp biodegradable absorbent comprises an absorbent capacity of up to about 490% by weight.

7. A process for producing a hemp biodegradable absorbent according to claim 1, wherein formulating the hemp biodegradable absorbent further comprises adding dust-reducing agents and fragrance.

8. A process for producing a hemp biodegradable absorbent according to claim 1, wherein formulating the hemp biodegradable absorbent further comprises pelletizing the hemp biomass.

9. A process for producing a hemp biodegradable absorbent according to claim 8, wherein pelletizing the hemp biomass comprises:
mixing a binding agent into the hemp biodegradable absorbent; and
forming a pellet from the mixed binding agent and hemp biodegradable absorbent.

10. A process for producing a hemp biodegradable absorbent according to claim 1, wherein formulating the hemp biodegradable absorbent further comprises combining the hemp biodegradable absorbent with a fertilizer blend.

11. A hemp biodegradable absorbent, comprising:
an absorbent material formed from an extracted hemp biomass without biomolecules and oleoresins and having a moisture content of less than about 10%, wherein the absorbent material comprises:
a particle size of the extracted hemp biomass of having a size range of between about 0.5 μm and about 50 μm; and
an absorbent capacity of between at least about 300% and up to about 600% by weight.

12. A hemp biodegradable absorbent according to claim 11, wherein a majority of the extracted hemp biomass comprises a particle size of about 0.5 μm or less.

13. A hemp biodegradable absorbent according to claim 11, wherein the hemp biodegradable absorbent comprises an absorbent capacity of up to about 490% by weight.

14. A hemp biodegradable absorbent according to claim 11, further comprising dust-reducing agents and fragrance.

15. A hemp biodegradable absorbent according to claim 11, wherein the absorbent material is pelletized.

16. A hemp biodegradable absorbent according to claim 15, wherein pelletized the hemp biomass comprises a binding agent mixed into the extracted hemp biomass.

17. A hemp biodegradable absorbent according to claim 11, further comprising a fertilizer blend mixed into the extracted hemp biomass.
